(19) United States
(12) Patent Application Publication
(10) Pub. No.: US 2026/0072018 A1
(43) Pub. Date: Mar. 12, 2026

(54) PROTEIN BINDING ASSAYS

(71) Applicant: NUCLERA LTD., Cambridge, Cambridgeshire (GB)

(72) Inventors: Michael Chun Hao Chen, Cambridge, Cambridgeshire (GB); Sihong Chen, Cambridge, Cambridgeshire (GB)

(21) Appl. No.: 18/871,846

(22) PCT Filed: Jun. 27, 2023
(86) PCT No.: PCT/GB2023/051671

§ 371 Date: Dec. 5, 2024

(30) Foreign Application Priority Data
Jun. 27, 2022 (GB) 2209358.7

(51) Int. Cl. G01N 33/542; G01N 21/64; G01N 33/68

(52) U.S. Cl. CPC G01N 33/542; G01N 21/6428; G01N 33/6845; G01N 2021/6439

(57) ABSTRACT

Provided herein are methods, and compositions for the synthesis of proteins. The methods are applicable to synthesis of proteins on a microfluidic device and assays using the expressed proteins.

### FIELD OF THE INVENTION

Provided herein are methods and compositions for the on-device expression and detection of protein synthesis and binding assays on the expressed proteins. The methods are applicable to monitoring protein binding assays on a microfluidic device.

### BACKGROUND TO THE INVENTION

The invention relates to methods for expression of proteins on a microfluidic device, particularly cell-free protein synthesis. Cell-free protein synthesis (CFPS) regimes are attractive alternatives to cell-based expression systems as they can be treated as reagents rather than organisms, making them amenable to in-vitro experimentation techniques. Additionally, cell-free systems are less sensitive to toxic protein synthesis; are open systems that can be modulated via addition of elements due to the lack of a cell membrane; are adaptable to high-throughput experiments; and can be used to good effect in small volumes. However, many of the cellular expression regulatory control paradigms still apply (e.g. incorrect ribosome binding motifs can lead to poor binding and poor transcription; incorrect codon usage can lead to inefficient translation etc).

Efficient protein synthesis relies on having the correct nucleic acid expression construct in the correct conditions. Protein synthesis and purification can be improved by attaching additional amino acids to the protein of interest, for example sequences improving solubility (solubility tags) or for purification (purification tags). In order to efficiently screen the optimal cell-free conditions for expression of a particular protein sequences, it is desirable to provide a population of nucleic acid expression constructs. Furthermore, in order to identify the best DNA construct to generate a protein of interest, it is desirable to provide a population of nucleic acid expression constructs. The invention herein describes methods for the preparation of nucleic acid constructs suitable for cell-free protein expression, and the use thereof.

When performing cell-free protein synthesis at microfluidic scale in a microfluidic device, such as a digital microfluidic device, it is useful to detect in real-time the proteins that are synthesized from said cell-free protein synthesis reaction. However, it is difficult to perform real-time detection of proteins in a cell-free protein synthesis reaction environment. The reaction contains many other proteins and biomolecules at high concentration, making non-specific protein detection via standard protein staining methods difficult (e.g., Coomassie Brilliant Blue G-250, SYPRO™ Ruby, Silver staining). Immunostaining or affinity-based purification followed by non-specific proteins staining are equally unhelpful as significant washing on a solid support must be performed to prevent background interference. As washing is known to be difficult in microfluidic and digital microfluidic devices, background interference may become debilitating.

Currently existing luminescent complementation approaches cannot achieve prolonged real-time detection in cell-free protein synthesis reactions, which often extend beyond 6 hours. This limit is due to a combination of reasons including O₂ consumption by the luminescence-generating enzyme that competes with cell-free protein synthesis O₂ requirements and temporary or permanent exhaustion of luminescent substrate over 3-24 hours of recombinant protein expression detection.

Proteins of interest may also be expressed as a fusion to a fluorescent protein, such as green fluorescent protein (GFP). However, GFP is a 26.9 kDa protein, which is the typical size for most fluorescent proteins. Tags of this size increase the total size of the protein of interest, especially if the protein of interest must be tagged with other large fusion proteins such as maltose-binding protein (MBP), which is 42.5 kDa. Given the average size of a human protein is ^{˜}52 kDa and the average size of an E. coli protein is ^{˜}35 kDa (Kim, Y. E. et al. Annu. Rev. Biochem. 2013. 82:323-355), the addition of a comparably sized fluorescent protein tag can significantly change the biological function and biophysical characteristics of a protein.

Many pieces of prior art disclose the use of sub-component tags for monitoring expression in a cellular system. For example U.S. Pat. No. 7,666,606 discloses protein-protein interaction detection systems using microdomains.

Schinn et al. Biotechnol. Bioeng 114 10 Oct. 2017 2412-2417. (https://onlinelibrary.wiley.com/doi/10.1002/bit.26305) discloses Rapid in-vitro screening for the location-dependent effects of unnatural amino acids on protein expression and activity—Schinn—2017—Biotechnology and Bioengineering—Wiley Online Library.

WO2022/038353 describes a method for measuring protein expression levels using split fluorescent protein systems. The level of expressed protein is measured in the presence of an excess of detector species, thereby measuring a single interaction which assembles a fluorescent protein.

### SUMMARY

Here we report the preparation of proteins in a droplet on a digital microfluidic device and binding assays using the expressed proteins. The assays can result in assembly of a fluorescent protein, which is detected in the device. The assays are performed by measuring the binding affinity in the presence of both the sub-components of the fluorescent proteins. Thus, protein species X and binding partner Y are modified with for example F₁₁ and F₁₋₁₀ where assembled F₁₋₁₁ is fluorescent. Despite both F₁₁ and F₁₋₁₀ being present, the concentrations are not high enough for them to interact and become fluorescent in the absence of X binding to Y. The interaction of X and Y allows formation of F₁₋₁₁ and the fluorescent signal generated is therefore a measure of the binding between X and Y.

Disclosed is a method for determining protein binding in a droplet on a digital microfluidic device having an array of electrodes comprising:
a. taking a droplet containing an expressed protein and a potential binding partner for the expressed protein, the expressed protein and binding partner each comprising sub-components of a fluorescent protein;
b. allowing the expressed protein and binding partner to bind, wherein the sub-components form a fully assembled fluorescent protein; and
c. determining the level of fluorescent signal within the droplets, thereby measuring the level of binding partner bound to the expressed protein.

Disclosed is a method for determining protein binding using assembly of a fluorescent protein in a droplet on a digital microfluidic device having an array of electrodes comprising:
a. taking a droplet containing an expressed protein (X) and a potential binding partner (Y) for the expressed protein, the expressed protein and binding partner each comprising sub-components of a fluorescent protein;
b. allowing the expressed protein (X) and binding partner (Y) to bind, wherein the sub-components also bind and form a fully assembled fluorescent protein when X binds to Y; and
c. determining the level of fluorescent signal within the droplets, thereby measuring the level of binding partner (Y) bound to the expressed protein (X).

Disclosed is a method for determining protein binding in a droplet on a digital microfluidic device having an array of electrodes comprising:
a. taking a droplet containing an expressed protein with a first fluorescent agent and a potential binding partner for the expressed protein, the potential binding partner having a second fluorescent agent; and
b. determining the level of colocalisation of the first and second fluorescent agents within the droplet, thereby measuring the level of binding partner bound to the expressed protein.

Any fluorescent protein may be used. The fluorescent protein may be sfGFP, GFP, eGFP, ccGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀. The peptide tag may be CFAST₁₁ or CFAST₁₀ and the further polypeptide CFAST in the presence of a hydroxybenzylidene rhodanine analog. The peptide tag may be ccGFP₁₁ and the further polypeptide ccGFP₁₋₁₀.

The fluorescent protein may be GFP. The fluorescent protein may be sfGFP. The fluorescent protein may be ccGFP.

The protein may be assembled and thereby become fluorescent as a result of the expressed protein binding with the binding partner. The affinity interaction results in the two sub-components of the fluorescent protein being near enough to each other to bind and induce fluorescence. Alternatively, each of the binding pairs can be labelled with a separate colour fluorophore. Upon binding the fluorophores become close together. A signal from either fluorescence energy transfer (FET) or colocalisation counting can determine the level of interaction between the expressed protein and the potential binding partner.

Thus the proximity based signal can either be from sub-component assembly, energy transfer or simply counting the colocalisation of two colours.

The fluorescent proteins or sub-components of the fluorescent protein may be attached to either the expressed protein (X) or the potential binding partner (Y). The expressed protein (X) may contain GFP₁₁, sfGFP₁₁ or ccGFP₁₁. The potential binding partner (Y) may be attached to GFP₁₋₁₀, sfGFP₁₋₁₀ or ccGFP₁₋₁₀.

The complementary ccGFP/GFP₁₁ peptide amino acid sequence could be the following:

1. KRDHMVLLEFVTAAGITGT
2. KRDHMVLHEFVTAAGITGT
3. KRDHMVLHESVNAAGIT
4. RDHMVLHEYVNAAGIT
5. GDAVQIQEHAVAKYFTV
6. GDTVQLQEHAVAKYFTV
7. GETIQLQEHAVAKYFTE

or a truncated version thereof. Truncations may involve a shortening of up to 5 amino acids from the N terminus, the C terminus or a combination thereof.

GFP₁₁ or GFP₁₋₁₀ can be fused to the protein of interest or binding partner through an amino acid linker. In one embodiment, the oligopeptide, peptide, or polypeptide linker can be 0-50 amino acids.

Expression can occur on or off the device. The expression may be performed in droplets on a digital microfluidic device having an array of electrodes.

Any binding partner can be assayed. The binding partner can be an amino acid sequence from a peptide or protein. Where the binding partner comprises an amino acid sequence, the binding partner can be expressed in droplets. The expression can be performed within the same droplet (co-expression) or within separate droplets that are then merged on the device.

Multiple proteins can be expressed and screened against multiple potential binding partners. For example, a population of (n) expressed proteins can be expressed in separate droplets, a population of (m) potential binding partners expressed in separate droplets and the droplets of expressed protein split into at least (m) number of droplets and the binding partners are each split into at least (n) number droplets and the droplets are combined to perform (n)×(m) number of binding assays simultaneously on the device.

Any expression system can be used to express the proteins. The transcription and translation may use a mammalian, insect, plant or bacterial derived expression system. The transcription and translation may use a human lysate system, a rabbit reticulocyte lysate (RRL) system, a Chinese Hamster Ovary (CHO) lysate system, a wheat germ cell-free system, a E. coli whole cell lysate system, a tobacco cell lysate system, a yeast lysate system or in a system of purified recombinant elements (PURE) or a mixture thereof. The reagents may be blended or supplemented to optimise expression.

The droplets are typically aqueous droplets in a hydrophobic oil layer. One or both of the layers may contain a surfactant. The surfactant in the oil layer may be a non-ionic surfactant. The surfactant in the oil layer may be a sorbitan ester such as Span85.

The use of the terms “in-vitro” and “cell-free” may be used interchangeably herein.

The expressed protein may be fused to multiple tags for detection, solubility and/or purification. For example the protein may be fused to multiple GFP₁₁ peptide tags and the synthesis occurs in the presence of multiple GFP₁₋₁₀ polypeptides. For example the protein may be fused to multiple sfCherry₁₁ peptide tags and the synthesis occurs in the presence of multiple sfCherry₁₋₁₀ polypeptides. The protein of interest may be fused to one or more sfCherry₁₁ peptide tags and one or more GFP₁₁ peptide tags and the synthesis occurs in the presence of one or more sfCherry₁₋₁₀ polypeptides and one or more GFP₁₋₁₀ polypeptides.

Any protein of interest may be synthesised. Any potential binding partner may be utilised.

The synthesis may be performed in a digital microfluidic device, for example an electrowetting-on-dielectric (EWoD) device.

### BRIEF DESCRIPTION OF THE DRAWINGS

FIG. 1: A schematic outlining the assay process.

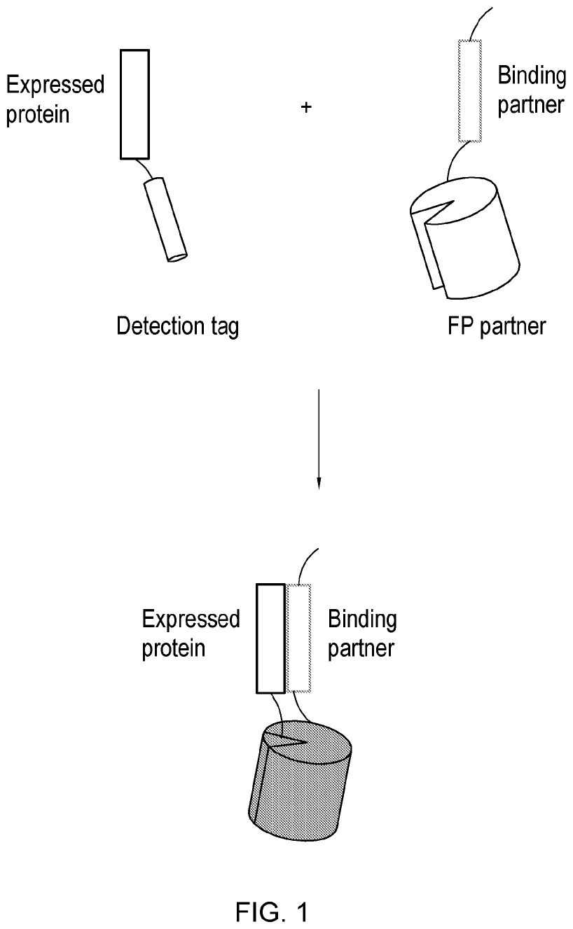

FIG. 2: In tube expression of proteins that bind and show fluorescent signal, along with proteins that do not bind and do not show fluorescent signal.

FIG. 3: On device expression of the proteins shown in FIG. 2. The droplets where binding is expected become fluorescent.

FIG. 4: Graphical representation of the fluorescent intensities from the droplets of FIG. 3.

FIG. 5: Coexpression of protein samples in tubes. High fluorescence is only seen when proteins bind. Where ccGFP is expressed as an expression control, high levels of signal are seen as no binding is required.

### DETAILED DESCRIPTION OF THE INVENTION

Disclosed is a method for determining protein binding in a droplet on a digital microfluidic device having an array of electrodes comprising:
a. taking a droplet containing an expressed protein and a potential binding partner for the expressed protein, the expressed protein and binding partner each comprising sub-components of a fluorescent protein;
b. allowing the expressed protein and binding partner to bind, wherein the sub-components form a fluorescent protein; and
c. determining the level of fluorescent signal within the droplets, thereby measuring the level of binding partner bound to the expressed protein.

Disclosed is a method for determining protein binding in a droplet on a digital microfluidic device having an array of electrodes comprising:
a. taking a droplet containing an expressed protein with a first fluorescent agent and a potential binding partner for the expressed protein, the potential binding partner having a second fluorescent agent; and
b. determining the level of colocalisation of the first and second fluorescent agents within the droplet, thereby measuring the level of binding partner bound to the expressed protein.

Disclosed is a method for determining protein binding using assembly of a fluorescent protein in a droplet on a digital microfluidic device having an array of electrodes comprising:
a. taking a droplet containing an expressed protein (X) and a potential binding partner (Y) for the expressed protein, the expressed protein and binding partner each comprising sub-components of a fluorescent protein;
b. allowing the expressed protein (X) and binding partner (Y) to bind, wherein the sub-components also bind and form a fluorescent protein when X binds to Y; and
c. determining the level of fluorescent signal within the droplets, thereby measuring the level of binding partner (Y) bound to the expressed protein (X).

Any fluorescent protein may be used. The fluorescent protein may be sfGFP, GFP, eGFP, ccGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀. The peptide tag may be CFAST₁₁ or CFAST₁₀ and the further polypeptide CFAST in the presence of a hydroxybenzylidene rhodanine analog. The peptide tag may be ccGFP₁₁ and the further polypeptide ccGFP₁₋₁₀.

The fluorescent protein may be GFP. The fluorescent protein may be sfGFP. The fluorescent protein may be ccGFP.

The protein may be assembled and thereby become fluorescent as a result of the expressed protein binding with the binding partner. The affinity interaction results in the two sub-components of the fluorescent protein being near enough to each other to bind and induce fluorescence. Alternatively each of the binding pairs can be labelled with a separate colour fluorophore. Upon binding the fluorophores become close together. A signal from either fluorescence energy transfer (FET) or colocalisation counting can determine the level of interaction between the expressed protein and the potential binding partner.

Thus the proximity based signal can either be from sub-component assembly, energy transfer or simply counting the colocalisation of two colours.

Exemplary assays are performed by measuring the binding affinity in the presence of both the sub-components of the fluorescent proteins. Thus protein species X and binding partner Y are modified with for example F₁₁ and F₁₋₁₀ where assembled F₁₋₁₁ is fluorescent. Despite both F₁₁ and F₁₋₁₀ being present, the concentrations are not high enough for them to interact and become fluorescent in the absence of X binding to Y. The interaction of X and Y allows formation of F₁₋₁₁ and the fluorescent signal generated is therefore a measure of the binding between X and Y.

Disclosed is a method for the cell-free expression of peptides or proteins in a digital microfluidic device. The droplets having the components required for cell-free protein synthesis (CFPS), otherwise known as in-vitro protein synthesis, can be manipulated by electrowetting in order to effect and improve protein expression.

Disclosed is a method for the cell-free expression of peptides or proteins in a digital microfluidic device. The droplets having the components required for cell-free protein synthesis (CFPS), otherwise known as in-vitro protein synthesis, can be manipulated by electrowetting in order to effect and improve protein expression.

The expressed protein can be any protein of interest. For example the expressed protein can be an antibody or part thereof. The expressed protein can be a nanobody, which lacks a light chain and are therefore significantly smaller than standard antibodies. Nanobodies have a high degree of flexibility at their antigen-binding interface. The screening of nanobodies with libraries of potential binding partners can be carried out if the nanobodies are expressed with a fluorescent protein or sub-component thereof attached.

Electrowetting is the modification of the wetting properties of a surface (which is typically hydrophobic) with an applied electric field. Microfluidic devices for manipulating droplets or magnetic beads based on electrowetting have been extensively described. In the case of droplets in channels this can be achieved by causing the droplets, for example in the presence of an immiscible carrier fluid, to travel through a microfluidic channel defined by the walls of a cartridge or microfluidic tubing. Embedded in the walls of the cartridge or tubing are electrodes covered with a dielectric layer each of which are connected to an A/C biasing circuit capable of being switched on and off rapidly at intervals to modify the electrowetting field characteristics of the layer. This gives rise to the ability to steer the droplet along a given path.

As an alternative to microfluidic channel systems, droplets can also be generated and manipulated on planar surfaces using digital microfluidics (DMF). In contrast to channel based microfluidics, DMF utilizes alternating currents on an electrode array for moving fluid on the surface of the array. Liquids can thus be moved on an open-plan device by electrowetting. Digital microfluidics allows precise control over the droplet movements including droplet fusion and separation.

Cell-free protein synthesis, also known as in-vitro protein synthesis or CFPS, is the production of peptides or proteins using biological machinery in a cell-free system, that is, without the use of living cells. The in-vitro protein synthesis environment is not constrained within a cell wall or limited by conditions necessary to maintain cell viability, and enables the rapid production of any desired protein from a nucleic acid template, usually plasmid DNA or RNA from an in-vitro transcription. CFPS has been known for decades, and many commercial systems are available.

Cell-free protein synthesis encompasses systems based on crude lysate (Cold Spring Harb Perspect Biol. 2016 December; 8(12): a023853) and systems based on reconstituted, purified molecular reagents, such as the PURE system for protein production (Methods Mol Biol. 2014; 1118: 275-284). CFPS requires significant concentrations of biomacromolecules, including DNA, RNA, proteins, polysaccharides, molecular crowding agents, and more (Febs Letters 2013, 2, 58, 261-268).

To date, digital microfluidics, electrowetting-on-dielectric (EWoD), and electrokinesis in general have only found limited uses in cell-free biological-based applications, mostly due to biofouling, where biological components such as proteins, nucleic acids, crude cell extracts and other bioproducts adsorb and/or denature to hydrophobic surfaces. Biofouling is well known in the art to limit the ability of EWoD devices to manipulate droplets containing biomacromolecules. Wheeler and colleagues report that the maximum actuation time for droplets on EWoD devices containing biological media is 30 min before biofouling inhibits EWoD-based droplet actuation (Langmuir 2011, 27, 13, 8586-8594).

Digital microfluidics can be carried out in an air-filled system where the liquid drops are manipulated on the surface in air. However, at elevated temperatures or over prolonged periods, the volatile aqueous droplets simply dry onto the surface by evaporation. This issue is compounded by the high surface area to volume ratio of nanoliter and microliter sized drops. Hence air-filled systems are generally not suitable for protein expression where the temperature of the system needs to be maintained at a temperature suitable for enzyme activity and the duration of the synthesis needs to be prolonged for synthesized proteins levels to be detectable.

Protein expression typically requires an ample supply of oxygen. The most convenient and high yielding way to power CFPS is via oxidative phosphorylation where O₂ serves as the final electron acceptor; however, there are other ways that involve replenishing with energy molecules not involved in oxidative phosphorylation. In a confined microfluidic or digital microfluidic system of droplets, insufficient oxygen is available to enable efficient protein synthesis.

Described herein are improved methods allowing for the cell-free expression of peptides or proteins in a digital microfluidic device. Included is a method for the cell-free expression of peptides or proteins in a microfluidic device wherein the method comprises one or more droplets containing a nucleic acid template (i.e., DNA or RNA) and a cell-free system having components for protein expression in an oil-filled environment, and moving said droplets using electrowetting. The components for the cell-free protein synthesis droplet can be pre-mixed prior to introduction to or mixed on the digital microfluidic device.

The droplet can be repeatedly moved for at least a period of 30 minutes whilst the protein is expressed. The droplet can be repeatedly moved for at least a period of two hours whilst the protein is expressed. The droplet can be repeatedly moved for at least a period of twelve hours whilst the protein is expressed. The act of moving the droplet allows oxygen to be supplied to the droplet and dispersed throughout the droplet. The act of moving improves the level of protein expression over a droplet which remains static.

The droplet can be moved using any means of electrowetting. The droplet can be moved using electrowetting-on-dielectric (EWoD). The electrical signal on the EWoD or optical EWoD device can be delivered through segmented electrodes, active-matrix thin-film transistors, or digital micromirrors.

The oil in the device can be any water immiscible liquid. The oil can be mineral oil, silicone oil such as dodecamethylpentasiloxane (DMPS), an alkyl-based solvent such as decane or dodecane, or a fluorinated oil. The oil can be oxygenated prior to or during the expression process. Alternatively, the device can be an air-filled device where droplets containing cell-free protein synthesis reagents are rapidly moved into position and fixed into an array under a humidified gas to prevent evaporation. Humidification can be achieved by enclosing or sealing the digital microfluidic device and providing on-board reagent reservoirs. Additionally, humidification can be achieved by connecting an aqueous reservoir to an enclosed or sealed digital microfluidic device. The aqueous reservoir can have a defined temperature or solute concentration in order to provide specific relative humidities (e.g., a saturated potassium sulfate solution at 30° C.).

A source of supplemental oxygen can be supplied to the droplets. For example droplets or gas bubbles containing gaseous or dissolved oxygen can be merged with the droplets during the protein expression. Additionally, a source of supplemental oxygen can be found by oxygenating the oil that is used as the filler medium. It is well-known in the art that oils such as hexadecane, HFE-7500, and others can be oxygenated to support the oxygen requirements of cell growth, especially E. coli cell growth (RSC Adv., 2017, 7, 40990-40995). Oxygenation can be achieved by aerating the oil with pure oxygen or atmospheric air.

The droplets can be formed before entering the microfluidic device and flowed into the device. Alternatively the droplets can be merged on the device. Included is a method comprising merging a first droplet containing a nucleic acid template such as a plasmid with a second droplet containing a cell-free extract having the components for protein expression to form a combined droplet capable of cell-free protein synthesis.

The droplets can be split on the device either before or after expression. Included herein is a method further comprising splitting the aqueous droplet into multiple droplets. If desired the split droplets can be screened with further additives. Included is a method wherein one or more of the split droplets are merged with the binding partner droplets for screening.

The cell-free expression of peptides or proteins can use a cell lysate having the reagents to enable protein expression. Common components of a cell-free reaction include an energy source, a supply of amino acids, cofactors such as magnesium, and the relevant enzymes. A cell extract is obtained by lysing the cell of interest and removing the cell walls, DNA genome, and other debris by centrifugation. The remains are the cell machinery including ribosomes, aminoacyl-tRNA synthetases, translation initiation and elongation factors, nucleases, etc. Once a suitable nucleic acid template is added, the nucleic acid template can be expressed as a peptide or protein using the cell derived expression machinery.

Any particular nucleic acid template can be expressed using the system described herein. Three types of nucleic acid templates used in CFPS include plasmids, linear expression templates (LETs), and mRNA. Plasmids are circular templates, which can be produced either in cells or synthetically. LETs can be made via PCR. While LETs are easier and faster to make, plasmid yields are usually higher in CFPS. mRNA can be produced through in-vitro transcription systems. The methods use a single nucleic acid template per droplet. The methods can use multiple droplets having a different nucleic acid template per droplet.

An energy source is an important part of a cell-free reaction. Usually, a separate mixture containing the needed energy source, along with a supply of amino acids, is added to the extract for the reaction. Common sources are phosphoenolpyruvate, acetyl phosphate, and creatine phosphate. The energy source can be replenished during the expression process by adding further reagents to the droplet during the process.

The cell-free extract having the components for protein expression includes everything required for protein expression apart from the nucleic acid template. Thus the term includes all the relevant ribosomes, enzymes, initiation factors, nucleotide monomers, amino acid monomers, metal ions and energy sources. Once the nucleic acid template is added, protein expression is initiated without further reagents being required.

Thus the cell-lysate can be supplemented with additional reagents prior to the template being added. The cell-free extract having the components for protein expression would typically be produced as a bulk reagent or ‘master mix’ which can be formulated into many identical droplets prior to the distinct template being separately added to separate droplets. Common cell extracts in use today are made from E. coli (ECE), rabbit reticulocytes (RRL), wheat germ (WGE), insect cells (ICE) and Yeast Kluyveromyces (the D2P system). All of these extracts are commercially available.

Rather than originating from a cell extract, the cell-free system can be assembled from the required reagents. Systems based on reconstituted, purified molecular reagents are commercially available, for example the PURE system for protein production, and can be used as supplied. The PURE system is composed of all the enzymes that are involved in transcription and translation, as well as highly purified 70S ribosomes. The protein synthesis reaction of the PURE system lacks proteases and ribonucleases, which are often present as undesired molecules in cell extracts.

The term digital microfluidic device refers to a device having a two-dimensional array of planar microelectrodes. The term excludes any devices simply having droplets in a flow of oil in a channel. The droplets are moved over the surface by electrokinetic forces by activation of particular electrodes. Upon activation of the electrodes the dielectric layer becomes less hydrophobic, thus causing the droplet to spread onto the surface. A digital microfluidic (DMF) device set-up is known in the art, and depends on the substrates used, the electrodes, the configuration of those electrodes, the use of a dielectric material, the thickness of that dielectric material, the hydrophobic layers, and the applied voltage. Once the CFPS reagents have been enclosed in the droplets, additional reagents can be supplied by merging the original droplet with a second droplet. The second droplet can carry any desired additional reagents, including for example oxygen or ‘power’ sources, or test reagents to which it is desired to expose to the expressed protein.

The droplets can be aqueous droplets. The droplets can contain an oil immiscible organic solvent such as for example DMSO. The droplets can be a mixture of water and solvent, providing the droplets do not dissolve into the bulk oil.

The droplets can be in a bulk oil layer. A dry gaseous environment simply dries the bubbles onto the surface during the expression process, leaving comet type smears of dried material by evaporation. Thus the device is filled with liquid for the expression process. Alternatively, the aqueous droplets can be in a humidified gaseous environment. A device filled with air can be sealed and humidified in order to provide an environment that reduces evaporation of CFPS droplets.

The droplets containing the cell-free extract having the components for protein expression will therefore typically be in the oil filled environment before the nucleic acid templates are added to the droplets. The templates can be added by merging droplets on the microfluidic device. Alternatively, the templates can be added to the droplets outside the device and then flowed into the device for the expression process. For example the expression process can be initiated on the device by increasing the temperature. The expression system typically operates optimally at temperatures above standard room temperatures, for example at or above 29° C.

The expression process typically takes many hours. Thus the process should be left for at least 30 minutes or 1 hour, typically at least 2 hours. Expression can be left for at least 12 hours. During the process of expression the droplets should be moved within the device. The moving improves the process by mixing the reagents and ensuring sufficient oxygen is available within the droplet. The moving can be continuous, or can be repeated with intervening periods of non-movement.

Thus the aqueous droplet can be repeatedly moved for at least a period of 30 minutes or one hour whilst the protein is expressed. The aqueous droplet can be repeatedly moved for at least a period of two hours whilst the protein is expressed. The aqueous droplet can be repeatedly moved for at least a period of twelve hours whilst the protein is expressed. The act of moving the droplet allows mixing within the droplet, and allows oxygen or other reagents to be supplied to the droplet. The act of moving improves the level of protein expression over a droplet which remains static.

Digital microfluidics (DMF) refers to a two-dimensional planar surface platform for lab-on-a-chip systems that is based upon the manipulation of microdroplets. Droplets can be dispensed, moved, stored, mixed, reacted, or analyzed on a platform with a set of insulated electrodes. Digital microfluidics can be used together with analytical analysis procedures such as mass spectrometry, colorimetry, electrochemical, and electrochemiluminescense.

The droplet can be moved using any means of electrowetting. The aqueous droplet can be moved using electrowetting-on-dielectric (EWoD). Electrowetting on a dielectric (EWoD) is a variant of the electrowetting phenomenon that is based on dielectric materials. During EWoD, a droplet of a conducting liquid is placed on a dielectric layer with insulating and hydrophobic properties. Upon activation of the electrodes the dielectric layer becomes less hydrophobic, thus causing the droplet to spread onto the surface.

The electrical signal on the EWoD or optically-activated amorphous silicon (a-Si) EWoD device can be delivered through segmented electrodes, active-matrix thin-film transistors or digital micromirrors. Optically-activated s-Si EWoD devices are well known in the art for actuating droplets (J. Adhes. Sci. Technol., 2012, 26, 1747-1771).

The oil in the device can be any water immiscible or hydrophobic liquid. The oil can be mineral oil, silicone oil such as dodecamethylpentasiloxane (DMPS), an alkyl-based solvent such as decane or dodecane, or a fluorinated oil. The air in the device can be any humidified gas.

A source of supplemental oxygen can be supplied to the droplets. For example droplets or gas bubbles containing gaseous or dissolved oxygen can be merged with the aqueous droplets during the protein expression. Alternatively the source of oxygen can be a molecular source which releases oxygen. Alternatively the droplets can be moved to an air/liquid boundary to enable increased diffusion of oxygen from a gaseous environment. Alternatively the oil can be oxygenated. Alternatively the droplets can be presented in a humidified air filled device.

Through an purification tag, such as a FLAG-tag, HIS-tag, GST-tag, MBP-tag, STREP-tag, or other form of purification tag, CFPS-expressed proteins can be immobilized to a solid-support resin and fresh batches of CFPS reagent can be delivered over the said resin. Thus, renewed reagents can be used to carry out protein synthesis, closely mimicking industrial methods of continuous flow (CF) and continuous exchange (CE) CFPS. By mimicking CF- and CE-CFPS, users can scale up their CFPS production methods.

The droplets can be actuated on a hydrophobic surface on the digital microfluidic device (ACS Nano 2018, 12, 6, 6050-6058). The hydrophobic surface can be a hydrophobic surface such as polytetrafluoroethylene (PTFE), Teflon AF (DuPont Inc), CYTOP (AGC Chemicals Inc), or FluoroPel (Cytonix LLC). The hydrophobic surface may be modified in such a way to reduce biofouling, especially biofouling resulting from exposure to CFPS reagents or nucleic acid reagents. The hydrophobic surface may also be superhydrophobic, such as NeverWet (NeverWet LLC) or Ultra-Ever Dry (Flotech Performance Systems Ltd). Superhydrophobic surfaces prevent biofouling compared with typical fluorocarbon-based hydrophobic surfaces. Superhydrophobic surfaces thus prolong the capability of digital microfluidic devices to move CFPS droplets and general solutions containing biopolymers (RSC Adv., 2017, 7, 49633-49648). The hydrophobic surface can also be a slippery liquid infused porous surface (SLIPS), which can be formed by infusing Krtox-103 oil (DuPont) with porous PTFE film (Lab Chip, 2019, 19, 2275).

Droplets can also contain additives to reduce the effects of biofouling on digital microfluidic surfaces. Specifically, droplets containing CFPS components can also contain additives such as surfactants or detergents to reduce the effects of biofouling on the hydrophobic or superhydrophobic surface of a digital microfluidic device (Langmuir 2011, 27, 13, 8586-8594). Such droplets may use antifouling additives such as TWEEN 20, Triton X-100, and/or Pluronic F127. Specifically, droplets containing CFPS components may contain TWEEN 20 at 0.1% v/v, Triton X-100 at 0.1% v/v, and/or Pluronic F127 at 0.05% w/v.

For electrowetting on dielectrics (EWoD), the change in contact angle of reagent upon the application of electric potential is an inverse function of surface tension. Thus, for low voltage EWoD operations, reduction in surface tension is achieved by addition of surfactants to reagents, which for CFPS reactions means to the lysate and to the DNA. This results in a dilution of the lysate, and it has been seen, in experiments, that diluting or otherwise adulterating the lysate results in a decrease in expression level of the protein of interest. Thus performing CFPS on DMF where the surfactants are added to the solutions being moved will necessarily result in a dilution and adulteration of the lysate and thus a decrease in the level of protein expression. In addition to being a problem in its own right, this further complicates extrapolation of on-DMF results to in-tube predictions of protein yield. An additional detriment of having to add surfactants to the samples is that this increases the time required for sample preparation, as well as increasing the potential for inconsistent results due to ‘user error,’ as there is more handling of reagents. An additional detriment of having to add surfactants to the samples is that certain downstream operations are hindered. For example, if a protein of interest is expressed in a cell-free system with a GFP₁₁ (or similar) peptide tag, it's downstream complementation with a GFP₁₋₁₀ (or similar) detector polypeptide is hindered in the presence of surfactant. Removal of the surfactant from the aqueous phase is therefore advantageous.

Rather than adding surfactants to the aqueous sample, it is instead possible to add surfactant, such as a sorbitan ester such as Span85 (e.g. Sorbitan trioleate, Sigma Aldrich, SKU 8401240025), to the oil. This has the advantages of enabling CFPS reactions to proceed on-DMF without dilution or adulteration. Additionally, it simplifies the sample preparation procedure for setting up the reactions, increasing the ease of use and the consistency of results. Using 1% w/w Span85 in dodecane allows for dilution-free CFPS reactions on-DMF, as well as dilution-free detection of the expressed non-fluorescent proteins. Other surfactants besides Span85, and oils other than dodecane could be used. A range of concentrations of Span85 could be used. Surfactants could be nonionic, anionic, cationic, amphoteric or a mixture thereof. Oils could be mineral oils or synthetic oils, including silicone oils, petroleum oils, and perfluorinated oils. Surfactants can have a detrimental effect on (1) the CFPS reactions and (2) the efficiency of the detection system (if the detection system involves complementation of a tag and detector). For example, by performing the CFPS reaction on-DMF with oil-surfactant mix, the detection of the expressed protein can also proceed without dilution and without adding aqueous surfactant. It has been shown that surfactants reduce the efficiency of some detection systems, including but not limited to the Split ccGFP (e.g. ccGFP₁₁/ccGFP₁₋₁₀) system, so removing surfactants from the reagent mix and instead adding them to the oil can be beneficial.

The peptide tag can be attached to the C or N terminus of the protein. The protein may be fused to multiple tags. For example the protein may be fused to multiple GFP₁₁ peptide tags and the synthesis occurs in the presence of multiple GFP₁₋₁₀ polypeptides. For example the protein may be fused to multiple sfCherry₁₁ peptide tags and the synthesis occurs in the presence of multiple sfCherry₁₋₁₀ polypeptides. The protein of interest may be fused to one or more sfCherry₁₁ peptide tags and one or more GFP₁₁ peptide tags and the synthesis occurs in the presence of one or more sfCherry₁₋₁₀ polypeptides and one or more GFP₁₋₁₀ polypeptides.

The complementary GFP₁₁ peptide amino acid sequence could be the following:

1. KRDHMVLLEFVTAAGITGT
2. KRDHMVLHEFVTAAGITGT
3. KRDHMVLHESVNAAGIT
4. RDHMVLHEYVNAAGIT
5. GDAVQIQEHAVAKYFTV
6. GDTVQLQEHAVAKYFTV
7. GETIQLQEHAVAKYFTE

or a truncated version thereof. Truncations may involve a shortening of up to 5 amino acids from the N terminus, the C terminus or a combination thereof.

GFP₁₁ or GFP₁₋₁₀ can be fused to the protein of interest or binding partner through an amino acid linker. In one embodiment, the oligopeptide, peptide, or polypeptide linker can be 0-50 amino acids.

Also disclosed are nucleic acid sequences for expressing particular tags. Nucleic acid sequences include

5′GGTGATACCGTTCAGCTGCAAGAACATGCAGTTGCAAAATACTTTACC
GTG
5′GGTGAAACCATCCAGTTACAAGAACACGCCGTGGCCAAATATTTCACC
GAA

These sequences may be repeated one or more times to produce a protein having multiple GFP₁₁ domains.

Purification tags may be appended to proteins so that they can be purified from their crude biological source using an affinity technique. The purification tags may be selected from for example FLAG-tag, His-tag, GST-tag, MBP-tag, STREP-tag. The Flag® tag, also known as the DYKDDDDK-tag, is a popular protein tag that is commonly used in affinity chromatography and protein research. His tags are polyhistidine strings of amino acids, typically between 6 and 9 histidine amino acids in length.

The binding moiety for the purification tags may contain four or more amino acids. The purification tags may contain 4-30 amino acids. The purification tags moiety may be selected from:

Alfa-tag (SRLEEELRRRLTE)
Avi-tag (GLNDIFEAQKIEWHE)
C-tag (EPEA)
Calmodulin-tag (KRRWKKNFIAVSAANRFKKISSSGAL)
Dogtag (DIPATYEFTDGKHYITNEPIPPK)
E-tag (GAPVPYPDPLEPR)
FLAG (DYKDDDDK)
G4T (EELLSKNYHLENEVARLKK)
HA (YPYDVPDYA)
His (HHHHHH)
Isopeptag (TDKDMTITFTNKKDAE)
lanthanide binding tag (LBT)(FIDTNNDGWIEGDELLLEEG)
Myc (EQKLISEEDL)
NE-Tag (TKENPRSNQEESYDDNES)
Poly Glutamate-tag (EEEEEEE)
Poly Arginine-tag (RRRRRRR)
Rho1D4-tag (TETSQVAPA)
SBP-tag (MDEKTTGWRGGHVVEGLAGELEQLRARLEHHPQGQREP)
Sdytag (DPIVMIDNDKPIT)
SH3 (STVPVAPPRRRRG)
SNAC (GSHHW)
Snooptag (KLGDIEFIKVNK)
Softag 1 (SLAELLNAGLGGS)
Softag 3 (TQDPSRVG)
Spot-tag (PDRVRAVSHWSS)
Spytag (AHIVMVDAYKPTK)
S-tag (KETAAAKFERQHMDS)
Strep-tag (AWAHPQPGG)(AWRHPQFGG)
Strep-tag II (WSHPQFEK)
T7tag (MASMTGGQQMG)
TC-tag (EVHTNQDPLD)
Ty-tag (CCPGCC)
VSV-tag (YTDIEMNRLGK)
Xpress-tag (DLYDDDDK)

The expressed protein may contain a sequence acting as a solubility enhancer, for example selected from:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation | IF2-domain 1 |
| factor IF2 | |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension | NEXT |
| sequence | |

The solubility tags may be selected from for example small ubiquitin related modifier (SUMO), maltose-binding protein (MBP), glutathione S-transferase (GST), Thioredoxin (TRX), Solubility eNhancing Ubiquitous Tag (SNUT) or FH8.

Disclosed is a method for determining protein binding in a droplet on a digital microfluidic device having an array of electrodes comprising:
a. taking a droplet containing an expressed protein and a potential binding partner for the expressed protein, the expressed protein and binding partner each comprising sub-components of a fluorescent protein, and wherein either the expressed protein or potential binding partner comprise a binding tag;
b. allowing the expressed protein and binding partner to bind, wherein the sub-components form a fluorescent protein;
c. determining the level of fluorescent signal within the droplets, thereby measuring the level of binding partner bound to the expressed protein;
d. capturing the fluorescent proteins via the binding tags;
e. moving the droplets using the electrodes, thereby removing the synthesised proteins from the droplet;
f. optionally washing the immobilised fluorescent proteins; and
g. optionally releasing the proteins and/or binding partners into further droplets.

### Multiplexed Binding Assays:

If for example the binding partners carry a different detection tags then the identity of the various partners can be detected. The binding partners may carry different coloured fluorophores or different tags for varying detector species. The colour the complex becomes indicates the binding partner as the fluorescence detected provides means of identifying the binding partner. The tag may be for example different amino acid sequences that form different coloured fluorescent proteins when assembled.

### Negative Binding Assays:

Where a known detectable binding partner Y is present, the assay can be disrupted by the presence of other ligands that bind to the partner X. The other ligands (Y′) can be unlabelled.

The assay measures the absence of the detectable X-Y species. Where the binding of known ligand X-Y is seen, the presence of Y′ has not displaced or inhibited the interaction of X and Y. The binding of X to Y′ causes a loss of signal. Therefore all droplets should be fluorescent apart from where the binding has been disrupted by other binding partners.

### Titration Assay

This technique involves serial dilution of protein X or protein Y to determine a saturating quantity of binding. The varying concentration of the reagents allows (or non-binding controls) immobilized on surface/beads and binding assessment of saturating quantity of Y. The assays may be used to determine the strength of binding for a particular ligand. The amount of protein X or Y can be controlled by varying the amount of protein expressed, the amount of binding or the volume of liquid used in various droplets. An alternative assay may be for example a assay comparing binding of a parent antibody or a modified antibody. If the two antibodies are labelled with different detector species, the binding of the modified or original antibodies can be identified. In such cases the immobilised sequences X could optionally varied to study binding of the two or more variants of Y. This method identifies if the original or modified antibody is a stronger binder, and to which species of X the two sequences bind.

### Devices

The manipulation of droplets by the application of electrical potential can be achieved on electrodes covered with an insulator or a dielectric or a series of insulators or dielectrics. Droplet manipulation as a result of an applied electrical potential is known as electrowetting. Electrowetting occurs as result of a non-uniform electric field that influences the hydrostatic equilibrium of a dielectric liquid (dielectrophoresis or DEP) or a change in the contact angle of the liquid on solid surface (electrowetting-on-dielectric or EWoD). DEP can also be used to create forces on polarizable particles to induce their movement. The electrical signal can be transmitted to a discrete electrode, a transistor, an array of transistors, or a sheet of semiconductor film whose electrical properties can be modulated by an optical signal.

EWoD phenomena occur when droplets are actuated between two parallel electrodes covered with a hydrophobic insulator or dielectric. The electric field at the electrode-electrolyte interface induces a change in the surface tension, which results in droplet motion as a result of a change in droplet contact angle. The electrowetting effect can be quantitatively treated using Young-Lippmann equation:

$$cos θ - cos {θ}_{0} = \{ 1 / 2 γ LG ) c \cdot {V}^{2}$$

where θ₀ is the contact angle when the electric field across the interfacial layer is zero, γLG is the liquid-gas tension, c is the specific capacitance (given as εᵣ. ε₀/t, where εᵣ is dielectric constant of the insulator/dielectric, so is permittivity of vacuum, t is thickness) and V is the applied voltage or electrical potential. The change in contact angle (inducing droplet movement) is thus a function of surface tension, electrical potential, dielectric thickness, and dielectric constant. When a droplet is actuated by EWoD, there are two opposing sets of forces that act upon it: an electrowetting force induced by electric field and resistant forces that include the drag forces resulting from the interaction of the droplet with filler medium and the contact line friction (ref). The minimum voltage applied to balance the electrowetting force with the sum of all drag forces (threshold voltage) is variably determined by the thickness-to-dielectric contact ratio of the insulator/dielectric, (t/εᵣ)^{1/2}. Thus, to reduce actuation voltage, it is required to reduce (t/εᵣ)^{1/2} (i.e., increase dielectric constant or decrease insulator/dielectric thickness). To achieve low voltage actuation, thin insulator/dielectric layers must be used. However, the deposition of high quality thin insulator/dielectric layers is a technical challenge, and these thin layers are easily damaged before the desired electrowetting contact angle is large enough to drive the droplet is achieved. Most academic studies thus report the use of much higher voltages >100V on easily fabricated, thick dielectric films (>3 μm) to effect electrowetting.

High voltage EWoD-based devices with thick dielectric films, however, have limited industrial applicability largely due to their limited droplet multiplexing capability. The use of low voltage devices including thin-film transistors (TFT) and optically-activated amorphous silicon layers (a-Si) have paved the way for the industrial adoption of EWoD-based devices due to their greater flexibility in addressing electrical signals in a highly multiplex fashion. The driving voltage for TFTs or optically-activated a-Si are low (typically <15 V). The bottleneck for fabrication and thus adoption of low voltage devices has been the technical challenge of depositing high quality, thin film insulators/dielectrics. Hence there has been a particular need for improving the fabrication and composition of thin film insulator/dielectric devices.

Typically, the electrodes (or the array elements) used for EWoD are covered with (i) a hydrophilic insulator/dielectric and a hydrophobic coating or (ii) a hydrophobic insulator/dielectric. Commonly used hydrophobic coatings comprise of fluoropolymers such as Teflon AF 1600 or CYTOP. The thickness of this material as a hydrophobic coating on the dielectric is typically <100 nm and can have defects in the form of pinholes or a porous structure; hence, it is particularly important that the insulator/dielectric is pinhole free to avoid electrical shorting. Teflon has also been used as an insulator/dielectric, but it has higher voltage requirements due to its low dielectric constant and the thickness required to make it pinhole free. Other hydrophobic insulator/dielectric materials can include polymer-based dielectrics such as those based on siloxane, epoxy (e.g. SU-8), or parylene (e.g., parylene N, parylene C, parylene D, or parylene HT). Due to minimal contact angle hysteresis and a higher contact angle with aqueous solutions, Teflon is still used as a hydrophobic topcoat on these insulator/dielectric polymers. However, there are difficulties in reliably producing <1 micron pinhole-free coatings of parylene or SU-8; thus, the thickness of these materials is typically kept at a 2-5 microns at the cost of increased voltage requirements for electrowetting. It has also been reported that traditional EWoD devices with parylene C are easily broken and unstable for repeated droplet manipulation with cell culture medium. Multi-layer insulator devices deposited with metal-oxide and parylene C films have been used to produce a more robust insulator/dielectric and enable operations with lower applied voltages. Inorganic materials, such metal oxides and semiconductor oxides, commonly used in the CMOS industry as “gate dielectrics”, have been used as insulator/dielectric for EWoD devices. They offer the advantage of utilizing standard cleanroom processes for thin film depositions (<100 nm). These materials are inherently hydrophilic, requiring an additional hydrophobic coating, and can be prone to pinhole formation as a result of thin film layer deposition process. Together with the need for lower voltage operations of EWoD, recent developmental work has focused on (1) using materials with improved dielectric properties (e.g., using high-dielectric constant insulators/dielectrics), (2) optimizing the fabrication process to make the insulator/dielectric pinhole free to avoid dielectric breakdown.

Operation of EWoD devices suffers from contact angle saturation and hysteresis, which is believed to be brought about by either one or combination of these phenomena: (1) entrapment of charges in the hydrophobic film or insulator/dielectric interface, (2) adsorption of ions, (3) thermodynamic contact angle instabilities, (4) dielectric breakdown of dielectric layer, (5) the electrode-electrode-insulator interface capacitance (arising from the double layer effect), and (6) fouling of the surface (such as by biomacromolecules). One of the adverse effects of this hysteresis is reduced operational lifetime of the EWoD-based device.

Contact angle hysteresis is believed to be a result of charge accumulation at the interface or within the hydrophobic insulator after several operations. The required actuation voltage increases due to this charging phenomenon resulting in eventual catastrophic dielectric breakdown. The most probable explanation is that pinholes at the insulator/dielectric may allow the liquid to come into contact with the electrode causing electrolysis. Electrolysis is further facilitated by pinhole-prone or porous hydrophobic insulators.

Most of the studies to understand contact angle hysteresis on EWoD have been conducted on short time scales and with low conductivity solutions. Long duration actuations (e.g., >1 hour) and high conductivity solutions (e.g., 1 M NaCl) could produce several effects other than electrolysis. The ions in solution can permeate through the hydrophobic coat (under the applied electric field) and interact with the underlying insulator/dielectric. Ion permeation can result in (1) change in dielectric constant due to charge entrapment (which is different from interfacial charging) and (2) change in surface potential of a pH sensitive metal oxide. Both can result in reduction of electrowetting forces to manipulate aqueous droplets, leading to contact angle hysteresis. The inventors have previously found that the damage from high conductivity solutions reduces or disables electrowetting on electrodes by inhibiting the modulation of contact angle when an electric field is applied.

An electrokinetic device includes a first substrate having a matrix of electrodes, wherein each of the matrix electrodes is coupled to a thin film transistor, and wherein the matrix electrodes are overcoated with a functional coating comprising: a dielectric layer in contact with the matrix electrodes, a conformal layer in contact with the dielectric layer, and a hydrophobic layer in contact with the conformal layer; a second substrate comprising a top electrode; a spacer disposed between the first substrate and the second substrate and defining an electrokinetic workspace; and a voltage source operatively coupled to the matrix electrodes.

The dielectric layer may comprise silicon dioxide, silicon oxynitride, silicon nitride, hafnium oxide, yttrium oxide, lanthanum oxide, titanium dioxide, aluminum oxide, tantalum oxide, hafnium silicate, zirconium oxide, zirconium silicate, barium titanate, lead zirconate titanate, strontium titanate, or barium strontium titanate. The dielectric layer may be between 10 nm and 100 μm thick. Combinations of more than one material may be used, and the dielectric layer may comprise more than one sublayer that may be of different materials.

The conformal layer may comprise a parylene, a siloxane, or an epoxy. It may be a thin protective parylene coating in between the insulating dielectric and the hydrophobic coating. Typically, parylene is used as a dielectric layer on simple devices. In this invention, the rationale for deposition of parylene is not to improve insulation/dielectric properties such as reduction in pinholes, but rather to act as a conformal layer between the dielectric and hydrophobic layers. The inventors find that parylene, as opposed to other similar insulating coatings of the same thickness such as PDMS (polydimethylsiloxane), prevent contact angle hysteresis caused by high conductivity solutions or solutions deviating from neutral pH for extended hours. The conformal layer may be between 10 nm and 100 μm thick.

The hydrophobic layer may comprise a fluoropolymer coating, fluorinated silane coating, manganese oxide polystyrene nanocomposite, zinc oxide polystyrene nanocomposite, precipitated calcium carbonate, carbon nanotube structure, silica nanocoating, or slippery liquid-infused porous coating.

The elements may comprise one or more of a plurality of array elements, each element containing an element circuit; discrete electrodes; a thin film semiconductor in which the electrical properties can be modulated by incident light; and a thin film photoconductor whose properties can be modulated by incident light.

The functional coating may include a dielectric layer comprising silicon nitride, a conformal layer comprising parylene, and a hydrophobic layer comprising an amorphous fluoropolymer. This has been found to be a particularly advantageous combination.

The electrokinetic device may include a controller to regulate a voltage provided to the individual matrix electrodes. The electrokinetic device may include a plurality of scan lines and a plurality of gate lines, wherein each of the thin film transistors is coupled to a scan line and a gate line, and the plurality of gate lines are operatively connected to the controller. This allows all the individual elements to be individually controlled.

The second substrate may also comprise a second hydrophobic layer disposed on the second electrode. The first and second substrates may be disposed so that the hydrophobic layer and the second hydrophobic layer face each other, thereby defining the electrokinetic workspace between the hydrophobic layers.

The method is particularly suitable for aqueous droplets with a volume of 1 μL or smaller.

The EWoD-based devices shown and described below are active matrix thin film transistor devices containing a thin film dielectric coating with a Teflon hydrophobic top coat. These devices are based on devices described in the E Ink Corp patent filing on “Digital microfluidic devices including dual substrate with thin-film transistors and capacitive sensing”, US patent application no 2019/0111433, incorporated herein by reference.

Described herein are electrokinetic devices, including:
a first substrate having a matrix of electrodes, wherein each of the matrix electrodes is coupled to a thin film transistor, and wherein the matrix electrodes are overcoated with a functional coating comprising:
a dielectric layer in contact with the matrix electrodes,
a conformal layer in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

Described herein is an electrokinetic device, including:
a first substrate having a matrix of electrodes, wherein each of the matrix electrodes is coupled to a thin film transistor, and wherein the matrix electrodes are overcoated with a functional coating comprising:
one or more dielectric layer(s) comprising silicon nitride, hafnium oxide or aluminum oxide in contact with the matrix electrodes,
a conformal layer comprising parylene in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

The electrokinetic devices as described may be used with other elements, such as for example devices for heating and cooling the device or reagent cartridges for the introduction of reagents as needed.

“Droplet” refers to a volume of liquid that electrowets a hydrophobic surface and is at least partially bounded by carrier fluid and/or, in some instances, a gas or gaseous mixture such as ambient air. For example, a droplet may be completely surrounded by carrier fluid or may be bounded by carrier fluid and one or more surfaces of an EWoD device. Droplets may take a wide variety of shapes; non-limiting examples include generally disc shaped, slug shaped, truncated sphere, ellipsoid, spherical, partially compressed sphere, hemispherical, ovoid, cylindrical, and various shapes formed during droplet operations, such as merging or splitting or formed as a result of contact of such shapes with one or more working surface of an EWoD device. Droplets may include typical polar fluids such as water, as is the case for aqueous or non-aqueous compositions, or may be mixtures or emulsions including aqueous and non-aqueous components. Droplets may also include dispersions and suspensions, for example magnetic beads in an aqueous solvent. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes.

“Droplet operation” refers to any manipulation of one or more droplets on a microfluidic device. A droplet operation may, for example, include: loading a droplet into the DMF device; dispensing one or more droplets from a source reservoir; splitting, separating or dividing a droplet into two or more droplets; moving a droplet from one location to another in any direction; merging or combining two or more droplets into a single droplet; diluting a droplet; mixing a droplet; agitating a droplet; deforming a droplet; holding a droplet in position; incubating a droplet; heating a droplet; vaporizing a droplet; cooling a droplet; disposing of a droplet; transporting a droplet out of a microfluidic device; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations includes but is not limited to microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles.

### EXAMPLES

Expression in tubes to generate fluorescent signals in the presence of binding species.

Sequences as shown below were used (Partner A):

| | | | Primary | |
|---|---|---|---|---|
| | | | target | |
| name | Protein Seq | Length aa | antigen | Link |
| N8-1 | QVQLQESGGGSVRPGESLRLSCIA | 111 | Tumor | http://www.sdab- |
| | AGTLFSGKAVGWYRQAPGKDRE | | protein p53 | db.ca/?Display& |
| | LVAALSSGRETYYIGPVKGRFIISR | | N-terminal | ID=sdAb_9422_ |
| | DDNKDTLYLQMNNLKLEDTAIYT | | transcription- | Sy |
| | CRLGLHWGQGTQVTVSS | | activation | |
| | | | domain | |
| N7-1 | QVQLQESGGGLAQAGESLRLSCV | 122 | Tumor | http://www.sdab- |
| | VSGRAFGNMAWFRQPPGKEREF | | protein p53 | db.ca/?Display& |
| | VGTIRANGERTYYSDSVKGRFTIS | | nuclear | ID=sdAb_7787_ |
| | RDNAKNSIYLQLNNLRPEDTAVY | | localization | Sy |
| | YCAARNSIQLFTSPREADYWGQG | | signaling | |
| | TQVTVSS | | (NLS) domain | |
| EGa1 | KYLLPTAAAGLLLLAAQPAMAQV | 169 | epidermal | http://www.sdab- |
| | QLQESGGGLVQPGGSLRLSCAAS | | growth | db.ca/?Search |
| | GRTFSSYAMGWFRQAPGKEREF | | factor | Results |
| | VAAIRWSGGYTYYTDSVKGRFTIS | | receptor | |
| | RDNAKTTVYLQMNSLKPEDTAVY | | (EGFR) | |
| | YCAATYLSSDYSRYALPQRPLDYD | | | |
| | YWGQGTQVTVSSAAAEQKLISEE | | | |
| | DLNGLEH | | | |
| 7D12 | AAQVKLEESGGGSVQTGGSLRLT | 126 | Human | http://www.sdab- |
| | CAASGRTSRSYGMGWFRQAPGK | | epidermal | db.ca/?Display& |
| | EREFVSGISWRGDSTGYADSVKG | | growth | ID=sdAb_4902_ |
| | RFTISRDNAKNTVDLQMNSLKPE | | factor | Ca |
| | DTAIYYCAAAAGSAWYGTLYEYD | | receptor | |
| | YWGQGTQVTVSS | | (HER1/EGFR) | |
| Anti-her2 | EVQLVESGGGLVQAGGSLRLSCA | 120 | Her2/ERBB2 | https://www.ncbi. |
| VHH | TSGITFMRYALGWYRQSPGKQRE | | Protein | nlm.nih.gov/pmc/ |
| | MVASINSGGTTNYADSVKGRFTI | | spike protein | articles/ |
| | SRDNAKNTVYLQMNSLKPEDTA | | | PMC6739820/pdf/ |
| | VYYCNARWVKPQFIDNNYWGQ | | | PRO-28-1865.pdf |
| | GTQVTVSS | | | |
| Anti-SARS | QVQLVESGGGLMQAGGSLRLSC | 126 | SARS-COV-2 | https://www. |
| RBD | AVSGRTFSTAAMGWFRQAPGKE | | receptor | nature.com/ |
| nanobody | REFVAAIRWSGGSAYYADSVKGR | | binding | articles/ |
| | FTISRDKAKNTVYLQMNSLKYEDT | | domain | s41594-020- |
| | AVYYCAQTHYVSYLLSDYATWPY | | (RBD) | 0469-6 |
| | DYWGQGTQVTVSSK | | | |

Binding partner A sequences were prepared to express a ccGFP₁₁ tag sequence attached to the amino acid sequences shown.

These bind to target proteins (B) as shown below:

| Protein | | | MW_POI |
|---|---|---|---|
| name | Protein seq | Length_aa | only (kDa) |
| TAD2 | LLPENNVLSPLPSQAMDDLMLSPDDIEQWFTEDPGPDEAPR | 45 | 5 |
| | MPEA | | |
| P53_ | PNNTSSSPQPKKKPLDGEYFTLQIRGRERFEMFRELNEALELK | 60 | 6.74 |
| tetramer | DAQAGKEPGGSRAHSSH | | |
| Her2 | NRPEDECVGEGLACHQLCARGHCWGPGPTQCVNCSQFLRG | 151 | 16.54 |
| (N498- | QECVEECRVLQGLPREYVNARHCLPCHPECQPQNGSVTCFG | | |
| A648) | PEADQCVACAHYKDPPFCVARCPSGVKPDLSYMPIWKFPDE | | |
| | EGACQPCPINCTHSCVDLDDKGCPAEQRA | | |
| SARS- | SVYAWNRKRISNCVADYSVLYNSASFSTFKCYGVSPTKLNDLC | 178 | 19.95 |
| CoV-2 | FTNVYADSFVIRGDEVRQIAPGQTGKIADYNYKLPDDFTGCVI | | |
| RBD | AWNSNNLDSKVGGNYNYLYRLFRKSNLKPFERDISTEIYQAG | | |
| | STPCNGVEGFNCYFPLQSYGFQPTNGVGYQPYRVVVLSFELL | | |
| | HAPATVCG | | |

The target B sequences were prepared using nucleic acid constructs in the format RBS/T7 promoter-seq B-ccGFP_{1-10_} terminator. All expressed protein sequence B strands also contain ccGFP₁₋₁₀.

The coexpression of Partner A samples was tested with Partner B samples. The samples of Partners A, based on literature analysis, are assumed to be able to bind with certain Partners B, and be unable to bind with other Partners B. Combinations were prepared as shown in the table below:

| Run | Partner A | Partner A name | Partner B | Partner B name |
|---|---|---|---|---|
| Run1 | A1 | N8-1 | B1 | TAD2 |
| | A1 | N8-1 | B2 | P53_tetramer |
| | A1 | N8-1 | B3 | Recep_L_domain1 |
| | A2 | N7-1 | B1 | TAD2 |
| | A2 | N7-1 | B2 | P53_tetramer |
| | A2 | N7-1 | B3 | Recep_L_domain1 |
| | A5 | [symbol] | B8 | [symbol] |
| | A6 | FERM | B8 | [symbol] |
| | A7 | PTB | B8 | [symbol] |
| | A8 | Anti-her2 VHH | B8_2 | Her2 (N498-A648) |
| | A8 | Anti-her2 VHH | B10 | Her2 (E2-T652) |
| | A8 | Anti-her2 VHH | B9 | The SARS-CoV-2 receptor binding domain |
| | | | | (RBD) of the spike protein ( [symbol] )(178aa) |
| | A9 | Anti-The (RBD) of the spike protein | B9 | The SARS-CoV-2 receptor binding domain |
| | | SARS-CoV-2 receptor binding domain | | (RBD) of the spike protein ( [symbol] )(178aa) |
| | A9 | Anti-The (RBD) of the spike protein | B8_2 | Her2 (N498-A648) |
| | | SARS-CoV-2 receptor binding domain | | |
| | A9 | Anti-The (RBD) of the spike protein | B10 | Her2 (E2-T652) |
| | | SARS-CoV-2 receptor binding domain | | |
| | Water | Water | Water | Water |
| | ccGFP | ccGFP | ccGFP | ccGFP |
| Run2 | A3 | EGa1 | B1 | TAD2 |
| | A3 | EGa1 | B2 | P53_tetramer |
| | A3 | [symbol] | B3 | Recep_L_domain1 |
| | A4 | 7D12 | B1 | TAD2 |
| | A4 | 7D12 | B2 | P53_tetramer |
| | A4 | 7D12 | B3 | Recep_L_domain1 |
| | Water | Water | Water | Water |
| | ccGFP | ccGFP | ccGFP | ccGFP |

[symbol]

indicates data missing or illegible when filed

24 nM DNA samples of respective Partners A and B were prepared in 96 well plates, and mixed with a reconstituted CFPS mix for protein expression. 2.5 μL of respective Partner A DNA, and 2.5 μL of respective Partner B DNA were mixed with 19 μL of expression mix (total volume of 24 μL per well). In addition, positive CFPS expression control (24 nM ccGFP DNA template) and negative CFPS control (water) were used as well.

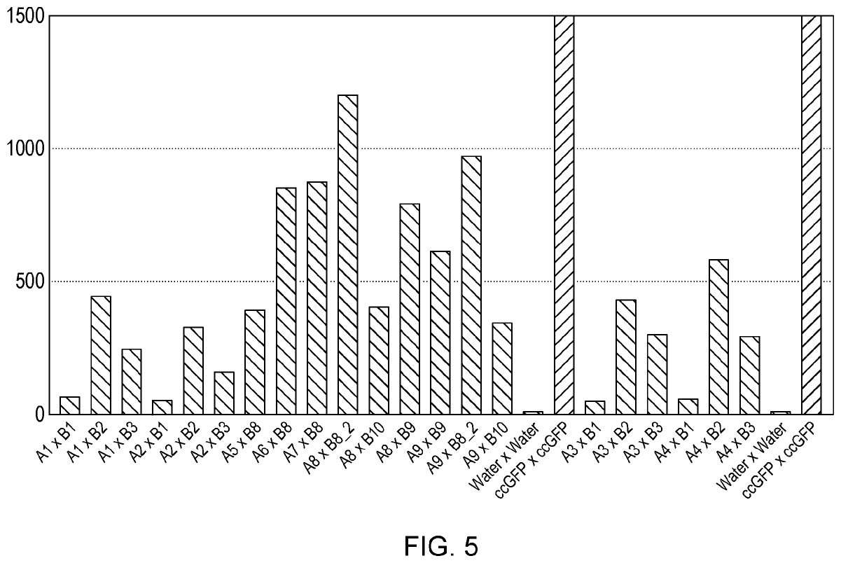

After the incubation was completed, 6 μL was taken from each corresponding well and transferred to 96 well plates with 54 μL buffer. After the samples were mixed well, 50 μL of mixtures were taken into black 384 well plates to be read with a fluorescence plate reader, shown in FIG. 5. From the data, the following partner A and B combinations were identified as positive binding control (PC): A1×B2, A2×B2, A7×B8, A8×B8_2, A8×B10, A9×B9, A3×B2, and A4×B2. Moreover, the combination A2×B1 was identified as negative binding control where no binding was seen (NC).

The binding (PC) and non-binding (NC) partners as shown below were taken for further evaluation:

| Partner A Name | Partner B Name | PRI Reaction |
|---|---|---|
| N8-1 | [symbol] tetramer | PC |
| N7-1 | | |
| [symbol] | | |
| 7D12 | | |
| N8-1 | TAD2 | NC |
| N7-1 | | |
| [symbol] | | |
| 7D12 | | |
| Anti-her2 VHH | Her2 ( [symbol] ) | PC |
| Anti-The (RBD) of the spike | The SARS-CoV-2 receptor | |
| protein_VHH1_H11-H4 | binding domain (RBD) of | |
| | the spike protein ( [symbol] ) | |
| Anti-her2 VHH | TAD2 | NC |
| Anti-The (RBD) of the spike | | |
| protein_VHH1_H11-H4 | | |

[symbol]

indicates data missing or illegible when filed

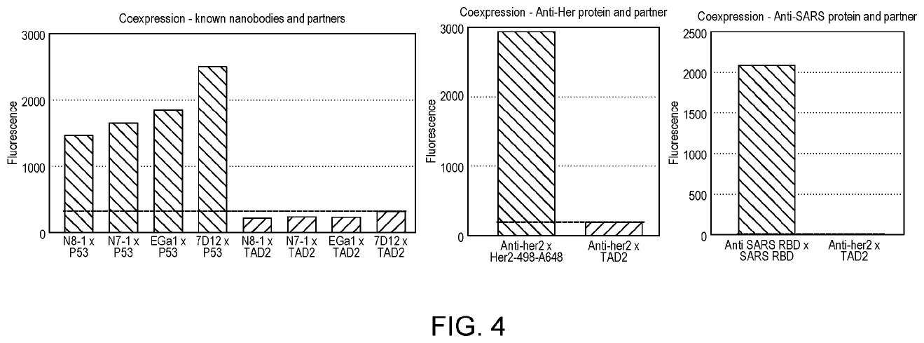

The co-expression was repeated as described above. Detailed data is shown in FIG. 4 for the pairs shown in the table. Where the two sequences bind, fluorescence is generated. Where the expressed partners do not bind, no fluorescence is seen.

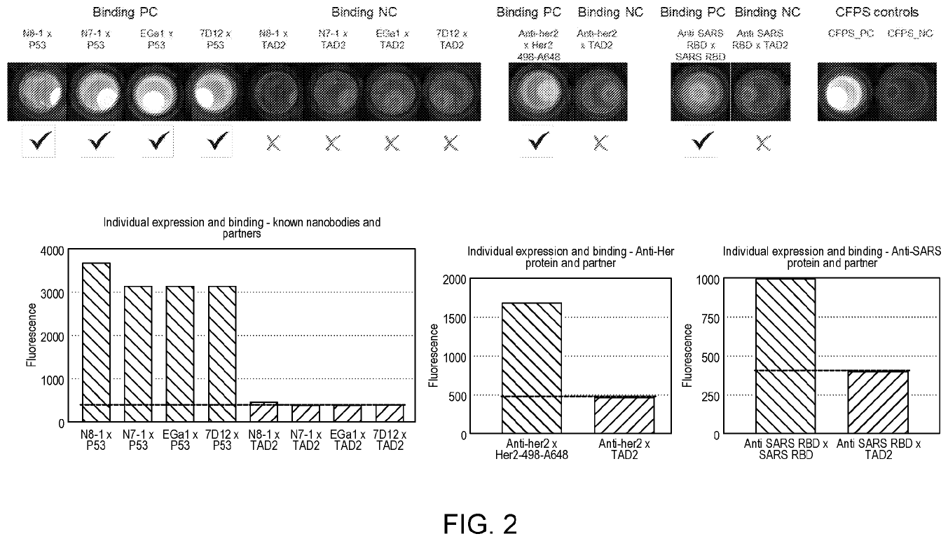

To validate the system, the proteins were also expressed separately and combined (data shown in FIG. 2). Equal amounts of sequences A and B were expressed and mixed together as below:

For individual expression and binding, the 24 nM DNA samples of respective Partners A and B were prepared in 96 well plates, and mixed with CFPS mix. 2.5 μL of respective Partner A DNA, and 2.5 μL of respective Partner B DNA were individually mixed with 9.5 μL of CFPS mix. A total volume of DNA and CFPS mixture was 12 μL per well. After the 5 hour incubation, the rows with Partner B CFPS mixtures were transferred into the corresponding Partner A rows in order to create the desired combination of Partner A and B. After the merge, Partner A wells contained CFPS mixtures from their own (Partner A), and their respective Partner B wells, making it a total volume of 24 μL per well.

After the incubation was completed, 6 μL was taken from each corresponding well and transferred to 96 well plates with 54 μL buffer. After the samples were mixed well, 50 μL of mixtures were taken into black 384 well plates to be read with a fluorescence plate reader, shown in FIG. 2.

### On-Device Coexpression

Reagents contains nucleic acid templates and expression mix were loaded onto an electrowetting device. The droplets were mixed and expression was performed at 29° C. for 4 hours. The samples, named above, were loaded into the cartridge, the samples layout in the cartridge can be seen in the table below:

| | Column number | | | | | | | |
|---|---|---|---|---|---|---|---|---|
| | 1 | 2 | 3 | 4 | 5 | 6 | 7 | 8 |
| Cartridge row A | [symbol] | [symbol] | [symbol] | [symbol] | [symbol] | [symbol] | [symbol] | [symbol] |
| Cartridge row B | [symbol] | [symbol] | [symbol] | [symbol] | [symbol] | [symbol] | [symbol] | [symbol] |

[symbol]

indicates data missing or illegible when filed

In this coexpression experiment, the template samples were mixed and expressed together (one combination per one droplet duplicated for two droplets). The combinations can be seen in the table below.

| | | | | | | | |
|---|---|---|---|---|---|---|---|
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |

[symbol]

indicates data missing or illegible when filed

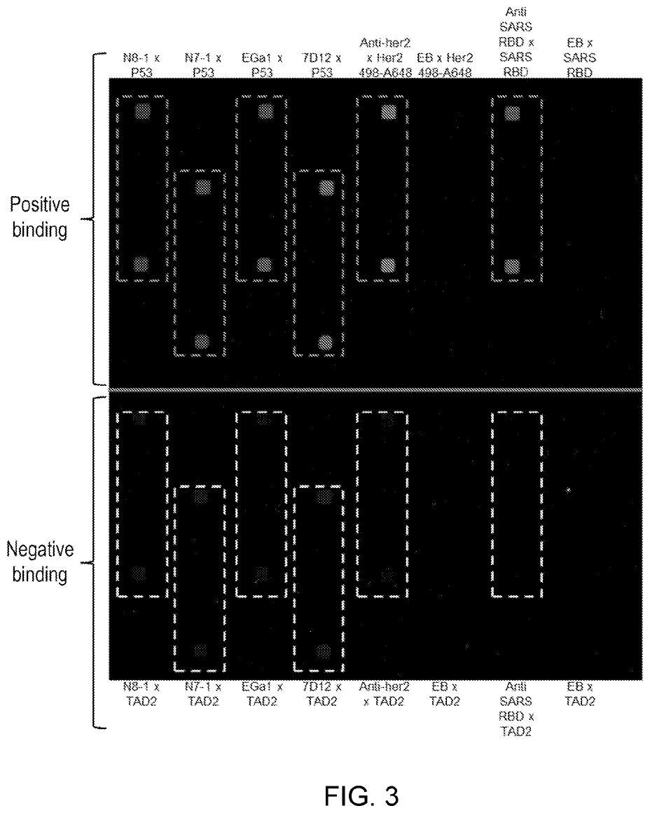

Droplets were both partners express and the proteins bind generate a fluorescent signal. Droplets where both partners express but the proteins do not bind do not generate a fluorescent signal. The fluorescent images are shown in FIG. 3, which a graphical representation in FIG. 4.

In summary, results confirmed the individual expression and binding of binding_PC Partner A and Partner B samples bind together very well and induce relatively high relative fluorescence unit reading values, while binding_NC Partner A and Partner B samples which do not bind well together induce much lower relative fluorescence unit reading values on electrowetting devices.

1. A method for determining protein binding using assembly of a fluorescent protein in a droplet on a digital microfluidic device having an array of electrodes comprising:
a. taking a droplet containing an expressed protein (X) and a potential binding partner (Y) for the expressed protein, the expressed protein and binding partner each comprising sub-components of a fluorescent protein;
b. allowing the expressed protein (X) and binding partner (Y) to bind, wherein the sub-components also bind and form a fluorescent protein when X binds to Y; and
c. determining the level of fluorescent signal within the droplets, thereby measuring the level of binding partner (Y) bound to the expressed protein (X).

2. The method according to claim 1, wherein the fluorescent protein is GFP.

3. The method according to claim 1, wherein the fluorescent protein is ccGFP.

4. The method according to claim 3, wherein the expressed protein contains ccGFP₁₁.

5. The method according to claim 3, wherein each potential binding partner is attached to ccGFP₁₋₁₀.

6. The method according to any one of claims 1 to 5, wherein the protein is expressed in droplets on a digital microfluidic device having an array of electrodes.

7. The method according to any one of claims 1 to 6, wherein the binding partner is an amino acid sequence.

8. The method according to claim 7, wherein the binding partner and fluorescent protein subcomponent are co-expressed in droplets on a digital microfluidic device having an array of electrodes.

9. The method according to any one of claims 1 to 8, wherein the expressed protein and binding partner are co-expressed in the same droplet.

10. The method according to any one of claims 1 to 9, wherein the expressed protein and binding partner are expressed in separate droplets which are merged on the device.

11. The method according to any one of claims 1 to 8, wherein a population of (n) expressed proteins are expressed in separate droplets, a population of (m) potential binding partners are expressed in separate droplets and the droplets of expressed protein is split into at least (m) number of droplets and binding partners are each split into at least (n) number droplets and the droplets are combined to perform (n)×(m) number of binding assays simultaneously on the device.

12. A method as claimed in any one of claims 1 to 11, wherein the transcription and translation occurs in human lysate system, a rabbit reticulocyte lysate (RRL) system, a Chinese Hamster Ovary (CHO) lysate system, a wheat germ cell-free system, a E. coli whole cell lysate system or in a system of purified recombinant elements (PURE) or a mixture thereof.

13. The method of any one of claims 1 to 12, wherein the droplets are in an oil layer and the oil layer contains surfactant.

14. The method of claim 13, wherein the surfactant in the oil layer is a non-ionic surfactant.

15. The method according to claim 14, wherein the surfactant is a sorbitan ester.

16. The method according to claim 15, wherein the surfactant is Span85.

17. The method according to any one preceding claim, wherein the assay contains a ligand which inhibits or displaces the binding of X and Y in order to determine the binding of the ligand by disrupting signal generated by the binding of X and Y.

18. The method according to any one preceding claim, wherein the assay determines the level of binding of two differently detectable species of partner Y.

19. The method according to claim 18, wherein a first species of Y contains a detectable tag comprising a sequence for a sub-component of first fluorescent protein and a second species of Y contains a detectable tag for a sub-component of a second fluorescent protein.


SEQUENCE LISTING (54 sequences)

SEQ ID NO: 1 - protein, 19 aa, synthetic construct
KRDHMVLLEFVTAAGITGT

SEQ ID NO: 2 - protein, 19 aa, synthetic construct
KRDHMVLHEFVTAAGITGT

SEQ ID NO: 3 - protein, 17 aa, synthetic construct
KRDHMVLHESVNAAGIT

SEQ ID NO: 4 - protein, 16 aa, synthetic construct
RDHMVLHEYVNAAGIT

SEQ ID NO: 5 - protein, 17 aa, synthetic construct
GDAVQIQEHAVAKYFTV

SEQ ID NO: 6 - protein, 17 aa, synthetic construct
GDTVQLQEHAVAKYFTV

SEQ ID NO: 7 - protein, 17 aa, synthetic construct
GETIQLQEHAVAKYFTE

SEQ ID NO: 8 - DNA, 51 bp, synthetic construct
  source 1..51: mol_type = other DNA
ggtgataccgttcagctgcaagaacatgcagttgcaaaatactttaccgtg

SEQ ID NO: 9 - DNA, 51 bp, synthetic construct
  source 1..51: mol_type = other DNA
ggtgaaaccatccagttacaagaacacgccgtggccaaatatttcaccgaa

SEQ ID NO: 10 - protein, 13 aa, synthetic construct
SRLEEELRRRLTE

SEQ ID NO: 11 - protein, 15 aa, synthetic construct
GLNDIFEAQKIEWHE

SEQ ID NO: 12 - protein, 4 aa, synthetic construct
EPEA

SEQ ID NO: 13 - protein, 26 aa, synthetic construct
KRRWKKNFIAVSAANRFKKISSSGAL

SEQ ID NO: 14 - protein, 23 aa, synthetic construct
DIPATYEFTDGKHYITNEPIPPK

SEQ ID NO: 15 - protein, 13 aa, synthetic construct
GAPVPYPDPLEPR

SEQ ID NO: 16 - protein, 8 aa, synthetic construct
DYKDDDDK

SEQ ID NO: 17 - protein, 19 aa, synthetic construct
EELLSKNYHLENEVARLKK

SEQ ID NO: 18 - protein, 9 aa, synthetic construct
YPYDVPDYA

SEQ ID NO: 19 - protein, 6 aa, synthetic construct
HHHHHH

SEQ ID NO: 20 - protein, 16 aa, synthetic construct
TDKDMTITFTNKKDAE

SEQ ID NO: 21 - protein, 20 aa, synthetic construct
FIDTNNDGWIEGDELLLEEG

SEQ ID NO: 22 - protein, 10 aa, synthetic construct
EQKLISEEDL

SEQ ID NO: 23 - protein, 18 aa, synthetic construct
TKENPRSNQEESYDDNES

SEQ ID NO: 24 - protein, 7 aa, synthetic construct
EEEEEEE

SEQ ID NO: 25 - protein, 7 aa, synthetic construct
RRRRRRR

SEQ ID NO: 26 - protein, 9 aa, synthetic construct
TETSQVAPA

SEQ ID NO: 27 - protein, 38 aa, synthetic construct
MDEKTTGWRGGHVVEGLAGELEQLRARLEHHPQGQREP

SEQ ID NO: 28 - protein, 13 aa, synthetic construct
DPIVMIDNDKPIT

SEQ ID NO: 29 - protein, 13 aa, synthetic construct
STVPVAPPRRRRG

SEQ ID NO: 30 - protein, 5 aa, synthetic construct
GSHHW

SEQ ID NO: 31 - protein, 12 aa, synthetic construct
KLGDIEFIKVNK

SEQ ID NO: 32 - protein, 13 aa, synthetic construct
SLAELLNAGLGGS

SEQ ID NO: 33 - protein, 8 aa, synthetic construct
TQDPSRVG

SEQ ID NO: 34 - protein, 12 aa, synthetic construct
PDRVRAVSHWSS

SEQ ID NO: 35 - protein, 13 aa, synthetic construct
AHIVMVDAYKPTK

SEQ ID NO: 36 - protein, 15 aa, synthetic construct
KETAAAKFERQHMDS

SEQ ID NO: 37 - protein, 9 aa, synthetic construct
AWAHPQPGG

SEQ ID NO: 38 - protein, 9 aa, synthetic construct
AWRHPQFGG

SEQ ID NO: 39 - protein, 8 aa, synthetic construct
WSHPQFEK

SEQ ID NO: 40 - protein, 11 aa, synthetic construct
MASMTGGQQMG

SEQ ID NO: 41 - protein, 10 aa, synthetic construct
EVHTNQDPLD

SEQ ID NO: 42 - protein, 6 aa, synthetic construct
CCPGCC

SEQ ID NO: 43 - protein, 11 aa, synthetic construct
YTDIEMNRLGK

SEQ ID NO: 44 - protein, 8 aa, synthetic construct
DLYDDDDK

SEQ ID NO: 45 - protein, 111 aa, synthetic construct
QVQLQESGGGSVRPGESLRLSCIAAGTLFSGKAVGWYRQAPGKDRELVAALSSGRETYYIGPVKGRFIISRDDNKDTLYLQMNNLKLEDTAIYTCRLGLHWGQGTQVTVSS

SEQ ID NO: 46 - protein, 122 aa, synthetic construct
QVQLQESGGGLAQAGESLRLSCVVSGRAFGNMAWFRQPPGKEREFVGTIRANGERTYYSDSVKGRFTISRDNAKNSIYLQLNNLRPEDTAVYYCAARNSIQLFTSPREADYWGQGTQVTVSS

SEQ ID NO: 47 - protein, 169 aa, synthetic construct
KYLLPTAAAGLLLLAAQPAMAQVQLQESGGGLVQPGGSLRLSCAASGRTFSSYAMGWFRQAPGKEREFVAAIRWSGGYTYYTDSVKGRFTISRDNAKTTVYLQMNSLKPEDTAVYYCAATYLSSDYSRYALPQRPLDYDYWGQGTQVTVSSAAAEQKLISEEDLNGLEH

SEQ ID NO: 48 - protein, 125 aa, synthetic construct
AAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVS

SEQ ID NO: 49 - protein, 120 aa, synthetic construct
EVQLVESGGGLVQAGGSLRLSCATSGITFMRYALGWYRQSPGKQREMVASINSGGTTNYADSVKGRFTISRDNAKNTVYLQMNSLKPEDTAVYYCNARWVKPQFIDNNYWGQGTQVTVSS

SEQ ID NO: 50 - protein, 128 aa, synthetic construct
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSK

SEQ ID NO: 51 - protein, 45 aa, synthetic construct
LLPENNVLSPLPSQAMDDLMLSPDDIEQWFTEDPGPDEAPRMPEA

SEQ ID NO: 52 - protein, 60 aa, synthetic construct
PNNTSSSPQPKKKPLDGEYFTLQIRGRERFEMFRELNEALELKDAQAGKEPGGSRAHSSH

SEQ ID NO: 53 - protein, 151 aa, synthetic construct
NRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRA

SEQ ID NO: 54 - protein, 178 aa, synthetic construct
SVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCG